(19) United States
(12) Patent Application Publication
(10) Pub. No.: US 2026/0151368 A1
(43) Pub. Date: Jun. 4, 2026

(54) Fertility Enhancement Composition

(71) Applicant: Exerkine Corporation, Hamilton (CA)

(72) Inventor: Mark Tarnopolsky, Hamilton (CA)

(21) Appl. No.: 19/126,848

(22) PCT Filed: Nov. 3, 2023
(86) PCT No.: PCT/CA2023/051474

§ 371 Date: May 2, 2025

Related U.S. Application Data
(60) Provisional application No. 63/382,192, filed on Nov. 3, 2022.

(51) Int. Cl. A61K 31/198; A61K 31/122; A61K 31/195; A61K 31/201; A61K 31/355; A61K 31/385; A61K 31/519; A61K 33/26; A61K 36/21; A61K 36/74; A61K 36/82; A61P 15/08

(52) U.S. Cl. CPC A61K 31/198; A61K 31/122; A61K 31/195; A61K 31/201; A61K 31/355; A61K 31/385; A61K 31/519; A61K 33/26; A61K 36/21; A61K 36/742; A61K 36/82; A61P 15/08

(57) ABSTRACT

A method for promoting fertility enhancement in a mammal is provided, comprising administering to the mammal a weight loss agent, a mitochondria-enhancing agent and a fertility support agent. Compositions for fertility enhancement are also provided.

### FIELD OF THE INVENTION

The present invention generally relates to a composition and method for improving fertility in mammals, including fertility in both males and females, age-related subfertility, and fertility in overweight or obese mammals.

### BACKGROUND OF THE INVENTION

Infertility affects ˜15% of couples worldwide. There has been an overall decline in fertility rates over the past 20 years in both men and women with several factors known or suggested. Factors such as aging and obesity/overweight are increasing significantly whilst fertility rates are declining and these factors are the best known to negatively influence fertility. Independent of the aforementioned factors, there are other factors such as smoking, alcohol intake, endocrine disruptors or factors of unknown origin that can negatively impact fertility.

The epidemic of obesity, or being overweight, has become one of the most significant health concerns faced at the global level. Over the past thirty years, the prevalence of obesity has almost tripled in number, to the extent that globally, approximately 40% of adults 18 years of age and older are now overweight and around 13% are obese. Recent data in the United States of America has found that the proportion of people who are overweight or obese (including severe obesity) has reached 70% for men and 67% for women. Obesity is caused by an imbalance between total energy expenditure and energy intake in an individual. In addition to other diseases associated with being overweight and obese (fatty liver disease, cardiovascular disease, type 2 diabetes, and dyslipidemia), it is known that being overweight and obese also predisposes an individual to infertility.

Epidemiological evidence suggests that excess bodyweight impairs both male and female fertility, reduces the efficacy of fertility treatments, and is linked to reproductive failure in a dose-dependent manner. Specifically, obesity is linked to erectile dysfunction, hypoandrogenism and altered spermatogenesis in men, and irregular menstruation, ovulatory dysfunction, hyperandrogenism and increased time to pregnancy in women, with a higher risk of birth defects, stillbirths and infant obesity in resultant neonates. Reproductive health is therefore a ‘couple concept’ because fertility of both partners is important for successful clinical pregnancies. Consequently, one aspect of the treatment of obesity is to induce weight loss through a reduction in body fat mass in both the male and female partner.

It is also well known that aging leads to a decline in the number and quality of oocytes in women, mainly driven by the hallmarks of biological aging (mitochondrial dysfunction, oxidative damage, inflammation, cell cycle arrest and apoptosis). Aging leads to a progressive loss of fertility in women until the complete cessation of menstruation at menopause at age 45-51 years in most countries. Women who are overweight and obese have an earlier age of menopause by ˜2 years. Other factors that contribute to early menopause include smoking and diabetes. An increasing number of women are delaying marriage and childbirth due to many socio-economic factors including; financial constraints, a longer time pursuing higher education and greater participation in the job market. This is placing a higher proportion of women in a higher age range where lower fertility is observed. There are a number of biological factors that contribute to ovarian aging including; mitochondrial dysfunction, oxidative stress, inflammation, cell-cycle arrest and apoptosis. Given that most women seeking advice for aging-associated infertility issues related to ovarian dysfunction are over age 30 y and have a finite timeline for treatment, it would be desirable to have a multi-ingredient supplement strategy that targets the pathophysiology of aging-associated ovarian dysfunction and that can be taken after age 30 y and for a shorter period of time.

Aging also alters the fertility of men due to a reduction in sex hormones, lower sperm count, DNA fragmentation and motility. These factors are also seen in higher frequency in younger men with infertility. Overall fertility and sperm parameters have been steadily dropping across all age groups over the past 20 years. The fundamental factors associated with low quality sperm (lower motility and number, DNA fragmentation, endocrine alterations) appear to have a similar biological basis as seen in ovarian dysfunction including; mitochondrial dysfunction, endocrine hormone disruption, oxidative stress, inflammation, cell-cycle arrest and apoptosis. Consequently, many studies have evaluated the potential for nutritional supplementation to enhance sperm parameters, mainly focusing on the use of single or combination anti-oxidants (Meta-analysis review; Salas-Huetos, A., et al., Adv Nutr. 9:833-, 2018). The latter review showed variable levels of efficacy on some aspects of sperm function in humans for coenzyme Q 10, alpha lipoic acid, vitamin C, vitamin E, zinc, omega-3 fatty acids and selenium but this data lacked consistency. Because of the high energy demands of sperm, some have suggested that optimal creatine-phosphocreatine cycling is required for optimal sperm function (Wallimann, T., et al, Amino Acids, 40:1271-, 2011). In agreement with this concept, one study found that low creatine content of sperm was associated with poor sperm quality (Nasrallah, F., et al., Clin Lab., 66:1, 2020). The aforementioned observations led to the speculation that creatine monohydrate supplementation would improve sperm quality (Ostojic, S., et al, Nutrients, 14:586, 2022). However, 6 weeks of creatine monohydrate supplementation (3 g/d) did not lead to an improvement in sperm quality in young men (Crow, M, et al., Int J Sport Med and Exerc Metab. 13:184-, 2003) and no other studies have been reported in a PUBMED search as of Nov. 3, 2023.

One of the consequences of obesity and aging is an increase in systemic and local inflammation. Obesity is also associated with mitochondrial dysfunction and this is one of the main drivers of obesity associated inflammation through the activation of the inflammasome pathway in diverse tissues (i.e., NL3P3, CASP1, IL-18, and IL-1B cell signalling. Obesity per se leads to an expansion of white adipose tissue, elevated free fatty acids levels, systemic inflammation (pro-inflammatory cytokines; IL-6, TNFα, IL-1B, and MCP-1), mitochondrial dysfunction, oxidative stress, insulin resistance and ectopic lipid deposition in insulin-sensitive tissues. Aging is also associated with chronic low-grade inflammation that is sometimes referred to an “inflammaging”, which is characterized by an increase in pro-inflammatory factors (as in obesity), inflammasome activation by cytokines (IL-1, IL-18, and CASP1), cell-cycle arrest (p16 and p21), and ultimately cell death (apoptosis). Aging may also cause a reduction in the protective anti-inflammatory factor levels, such as IL-10, in both tissues and in circulation. Collectively, these inflammatory processes can alter the neuroendocrine and reproductive systems, causing hypothalamic-pituitary-adrenal axis dysregulation, thereby impairing gonadal function and fertility.

Obesity, polycystic ovarian syndrome (PCOS), aging, and other unknown causes of subfertility in women are associated with fewer viable and growing ovarian follicles, an increase in atretic follicles and an increase in the number of apoptotic granulosa cells. Strategies that can reverse the aforementioned ovarian pathologies caused by aging, obesity, PCOS or unknown factors, and thus mitigate the molecular root-causes of female subfertility (e.g., mitochondrial dysfunction, oxidative stress, apoptosis, cell-cycle arrest and inflammation) should be developed to result in an improvement in successful pregnancies (copulatory success).

Obesity, aging and unknown factors may cause male subfertility that is associated with testicular pathology, including sperm cell-cycle arrest and apoptotic seminiferous tubules. Strategies that can reverse male subfertility driven by aging, obesity or unknown factors and thereby mitigate the molecular root causes in testicles (mitochondrial dysfunction, oxidative stress, inflammation, cell-cycle arrest, apoptosis and/or reduced energy state (ATP and phosphocreatine levels), should lead to a greater likelihood of successful pregnancies (copulatory success).

To date there have been many suggestions for individual or combination nutritional supplement strategies that have shown some evidence of efficacy in either targeting specific aspects of cellular pathology in male or female sex organs and/or enhancing functional outcomes (i.e., sperm quality, pregnancy success, etc.); however, no single multi-ingredient supplement strategy has been shown to be effective at targeting the cellular, molecular, structural and functional capacity of the sex organs across all clinical spectra (obesity/overweight, aging and in younger healthy mammals).

It would be desirable, thus, to provide a fertility enhancement (FE) composition and a method for improving the fecundity/copulatory success of overweight and obese individuals, aging adults with subfertility, as well as healthy men and women.

### SUMMARY OF THE INVENTION

A nutrient fertility enhancer (FE) composition/kit has now been developed which has been determined to enhance the fertility/copulatory success of male and female mammals, including those with infertility or subfertility.

Thus, in a first aspect of the present invention, a fertility enhancement composition comprising a combination of weight loss and mitochondria-enhancing agents, combined with fertility support agents is provided.

In one embodiment, a fertility enhancement (FE) composition comprising weight loss agents, mitochondria-enhancing agents and fertility support agents is provided, wherein the weight loss agents comprise green tea extract, green coffee bean extract and forskolin, the mitochondria-enhancing agents comprise beet root extract, coenzyme Q10, alpha lipoic acid, creatine monohydrate and vitamin E, and the fertility support agents comprise L-arginine, iron, folate/folic acid and omega-3 fatty acids such as docosahexanoic and/or ecosapentanoic acid.

In another aspect of the invention, a method is provided for promoting fertility enhancement (FE) in a mammal comprising the step of administering to the mammal a fertility enhancement composition comprising weight loss agents and mitochondria-enhancing agents, together with a combination of supplements designed to support fertility and/or mitigate aspects of infertility (fertility support agents) in male and female mammals.

In a further aspect, a method of treating infertility in a mammalian male and female couple is provided comprising: i) administering to the male of the couple a fertility-enhancement composition as defined above; ii) administering to the female of the couple a fertility-enhancement composition as defined above; or iii) administering to both the male and female of the couple a fertility-enhancement composition as defined above.

These and other aspects of the present invention will become apparent in the detailed description that follows, by reference to the following figures.

### BRIEF DESCRIPTION OF THE FIGURES

FIG. 1 graphically illustrates A) total copulatory plugs and B) daily copulatory plugs during mating period. LF=Low Fat Diet, HF=Western Diet (WD), and FE=‘Fertility Enhancer’ multi-nutrient supplement with WD. *Significantly different from LF (p≤0.05). § is significantly different from HF (p≤0.05).

FIG. 2 graphically illustrates A) total litter number and B) copulatory success following gestation. LF=Low Fat Diet, HF=Western Diet (WD), and FE=‘Fertility Enhancer’ multi-nutrient supplement with WD. *Significantly different from LF (p≤0.05). § is significantly different from HF (p≤0.05).

FIG. 3 graphically illustrates A) growing follicle counts per ovary and B) growing follicle counts per mm³. Growing follicles=all follicles counted excluding primordial, atretic and corpora lutea. LF=Low Fat Diet, HF=Western Diet (WD), and FE=‘Fertility Enhancer supplement with WD. *Significantly different from LF (p≤0.05). § is significantly different from HF (p≤0.05).

FIG. 4 graphically illustrates atretic follicle counts A) per ovary and B) per mm³. LF=Low Fat Diet, HF=Western Diet (WD), and FE=‘Fertility Enhancer’ supplement with WD). Follicles containing dark, pyknotic nuclei within the granulosa cells were considered atretic. *Significantly different from LF (p≤0.05). § is significantly different from HF (p≤0.05).

FIG. 5 graphically illustrates the mRNA abundance for markers of inflammation, A) IL-1B, B) Casp1, and C) TNFα, in ovarian tissue showing higher abundance for IL-1B and Casp1 for the high-fat (HF) vs the low-fat (LF) and fertility-enhancement (FE) groups and lower TNFα for the FE vs HF group.

FIG. 6 graphically illustrates that the level of markers of inflammation seen in livers of both male and female HF animals with an FE diet were similar to LF animals, including A) TNFα, B) IL1b, C) CD86, D) Casp1, and E) NLRP3. Collectively, the data show less systemic inflammation (liver) with the FE diet in both males and females and less ovarian inflammation in the females on the FE diet vs HF alone diet. Unexpectedly, the results also demonstrate a reduction in both systemic mitochondrial associated inflammasome activation (IL-1B, Casp1, NLRP3) and general inflammation (TNFα, CD86) for the FE diet vs the HF animals.

FIG. 7 graphically illustrates body weights in young (Y) and old (O) mice fed either the control (CON) or the Fertility Enhancer (FE) diet for 5 weeks, including A) body weight change over time, and B) final body weights. Collectively, these data demonstrate that the fertility enhancement composition improves bodyweights across age groups.

FIG. 8 graphically illustrates A) total fat mass, B) gonadal white adipose tissue (WAT) fat mass and C) skeletal muscle mass in young and old mice following 5 weeks of dietary intervention and after two consecutive breeding periods. These data clearly show that the fertility enhancement composition improves body composition (lower total fat mass), decreases gonadal fat mass and increases muscle mass in both younger and older mice.

FIG. 9 graphically illustrates the effect on lipid oxidation in young and old female of a control or FE diet. This clearly shows that the fertility enhancement composition improves whole-body fat oxidation in both younger and older mice.

FIG. 10 graphically illustrates the effect on number of litters following two consecutive breeding periods in young and old mice following 5 weeks of dietary intervention with either the control or FE diet. This figure demonstrates that the fertility enhancement composition improves fertility in female mice as indicated by the increased number of litters in both younger and older cohorts after a breeding period of A) 5 days; B) 10 days and C) 15 days.

FIG. 11 graphically illustrates the effect on ovarian A) oxidative damage (4-HNE), B) mitochondrial antioxidant defense (SOD 2), C) inflammation (CASP1) and D) inflammation (IL-18) in the older female mice. These data prove that the fertility enhancement composition targets the major driving hallmarks of age-induced infertility, including oxidative stress/damage, mitochondrial dysfunction (antioxidants status), and inflammation in ovarian tissue.

FIG. 12 graphically illustrates the effect on testicular A) anti-inflammatory factors (IL-10), key inhibitors of cell-cycle arrest B) p16 and C) p21 in younger male mice. It also shows remarkable effects on body composition: D) total fat mass, E) percent body fat and F) the body composition index (lean mass/fat mass). These data demonstrate that the fertility enhancer composition improves body composition, anti-inflammatory factors and cell-cycle arrest; thus, likely favouring overall health and the maturation of sperm cells/spermatogenesis in testicles of younger males.

### DETAILED DESCRIPTION OF THE INVENTION

A fertility enhancement (FE) composition comprising; weight loss agents, mitochondria-enhancing agents, and optionally fertility-support agents, is provided which is useful to enhance fertility and/or improve reproductive success in mammals, including both male and female mammals, young and aging mammals, and overweight or obese mammals.

The term “fertility” refers to the ability to reproduce, i.e. a mammalian couple of reproductive age that is able to conceive and achieve a viable pregnancy within a given period of time, for example, a period of about 12 months, of regular, unprotected sexual intercourse. “Subfertility” is a failure to conceive naturally after 12 months of unprotected regular sexual intercourse. “Infertility” is the failure to conceive naturally without medical intervention.

The term “fertility enhancement” is used herein to refer to a treatment that promotes fertility and increases the potential for a mammal to conceive and achieve a viable pregnancy within a given period of time. Thus, fertility enhancement may include one or more of increasing or maintaining sperm quality and/or function (volume, motility, count, concentration, morphology and DNA integrity), oocyte and embryo quality, and pregnancy and fertilization rate. Fertility enhancement may also include achieving, progressing towards or maintaining one or more of: a healthy body weight, healthy body fat levels, a higher body composition index, healthy mitochondrial function, reduced systemic and/or sex organ/gonadal specific inflammation, reduced oxidative stress and lesser gonadal tissue apoptosis levels.

With respect to body weight, one of the most widely used metrics for measuring body weight is the “body mass index” or BMI. A BMI range of about 18.5 to 24.9 is considered to be the normal healthy range for the BMI index, e.g. a body weight within a range that promotes the health, fitness, well-being and physical appearance of an individual. Overweight and obesity are defined as the abnormal or excessive accumulation of fat that may cause health impairments. An individual having a BMI between about 25-29.9 is generally considered to be overweight, while an individual having a BMI equal to or greater than 30 is generally considered to be obese. The term “weight loss” is used herein to refer to achieving, progressing towards or maintaining a body weight within a normal healthy range.

Regarding a healthy range of body fat levels, e.g. a body fat percentage within a range that promotes the health, fitness, well-being and physical appearance of an individual, an example of a healthy body fat range is an amount ranging from the essential body fat level (i.e. the amount considered essential for physical and psychological well-being up to approximately 20% above the essential body fat level. According to the American Council on Exercise, the amount of fat considered to be essential for men is about 2-5% body fat and for women is about 10-13% body fat.

Given the importance of achieving a lower body fat while maintaining lean body mass (mainly skeletal muscle), it is important to consider both of these variables together when evaluating changes in body composition. These variables cannot be assessed by body weight or body mass index (BMI) alone, and thus, these can be misleading concepts when considering weight loss. A novel concept that expresses the relationship between fat and lean mass more accurately is the “body composition index” (BCI), the ratio of total lean mass/total fat mass (in units of weight) with higher or increasing ratios reflecting a more optimal body composition (greater muscle and/or less fat).

The fertility enhancement (FE) composition comprises at least one weight loss agent. The weight loss agent may be any suitable agent that possesses the characteristic of promoting weight loss (and/or little or no loss of lean mass), e.g. maintaining a healthy body weight (BCI), in a mammal, e.g. by at least about 0.5%, and preferably a body weight reduction, or BCI increase, of about 1% or more, e.g. by 5%, 10%, 20%, or greater of body weight or BCI in the individual prior to treatment with the present composition, or a reduction in the rate of rising body weight or declining BCI by at least about 1% or more, and preferably a reduction of about 5% or more, e.g. by 10%, 20%, or greater to the rate of body weight increase or BCI decrease prior to treatment, e.g. by at least about 0.5%, and preferably a body weight reduction of about 1% or more, e.g. by 5%, 10%, 20%, 25%, 30%, 40%, or greater, of body weight of the individual prior to treatment with the present composition, or a reduction in the rate of rising body weight by at least about 1% or more, and preferably a reduction of about 5% or more, e.g. by 10%, 30%, 50%, 70%, 90% or greater to the rate of body weight increase prior to treatment.

Suitable weight loss agents may promote weight loss or maintenance of a healthy body weight by any one or more of several mechanisms including, but not limited to, the following: decreasing food intake or promoting satiety, decreasing lipid absorption, increasing energy expenditure, decreasing pre-adipocyte differentiation and proliferation, decreasing lipogenesis or increasing lipolysis or lipid oxidation. Examples of weight loss agents include, but are not limited to; chitosan, psyllium, guar gum, capsaicin, caffeine, Garcina cambogia, Pinus densiflora, capsaicin, yohimbe, hoodia, glucomannan, African mango, guarana, pyruvate, carnitine, beta-glucans, fucoxanthin, raspberry ketone, white kidney bean, kola nut, chromium, ginseng, psyllium, St. John's wort, dandelion, hydroxycitric acid, conjugated linoleic acid, green tea extract, black tea extract, green coffee bean extract, forskolin, bitter orange and mixtures thereof.

In one embodiment, the fertility enhancement composition comprises a mixture of green tea extract, green coffee bean extract and forskolin as weight loss agents.

Green tea extract for use in the present composition may be selected from any suitable source, including green tea leaf or other green tea source such as Sencha, Fukamushi Sencha, Gyokuro, Kabusecha, Matcha, Tencha, Genmaicha, Matcha, Shincha, Hojicha, Ichibanchagreen, Nibancha and Sanbancha tea, which are derived from the Camellia sinensis leaf Green tea is abundant in polyphenols such as catechins. Examples of such catechins include epigallocatechin gallate, catechin, catechin gallate, epicatechin, gallocatechin, epigallocatechin, and epicatechin gallate. Preferably, the green tea extract for use in the composition comprises about 80% or more of catechins by dry weight, with about 50% or more of the catechins being epigallocatechin gallate. Green tea extract for use in the present composition may be either caffeinated or substantially decaffeinated, for example, having less than 1% caffeine by dry weight. In one embodiment, the green tea extract comprises about 0.05-90% of the dry weight of the fertility enhancement composition, such as about 5-50%, or about 10-30% of the dry weight of the composition. In another embodiment, the fertility enhancement composition comprises a daily dosage of about 10 mg-5 g of green tea extract and preferably, about 25-1000 mg, for example, 50-500 mg/day, 100-300 mg/day or 250 mg/day.

Green coffee bean extract for use in the present composition may be selected from any suitable green coffee bean source such as Coffea Arabica or Coffea canephora. Green coffee beans contain several types of chlorogenic acids, such as 3-caffeoylquinic acid, 4-caffeoylquinic acid and 5-caffeoylquinic acid. Preferably, the green coffee bean extract for use in the present composition comprises about 30% or more of chlorogenic acids by dry weight. Green coffee bean extract for use in the present composition may be either caffeinated or substantially decaffeinated, for example, having less than 1% of caffeine by dry weight. Preferably the green coffee bean extract comprises at least 35% chlorogenic acids and at least 35% caffeine by dry weight. In one embodiment, the green coffee bean extract comprises about 0.1-80% of the dry weight of the fertility enhancement composition, such as about 5-50%, or about 10-30% of the dry weight of the composition. In another embodiment, the fertility enhancement composition comprises a daily dosage of about 10 mg-5 g of green coffee bean extract and preferably, about 50-1000 mg, for example, 50-500 mg/day, 100-300 mg/day or 250 mg/day.

Forskolin for use in the present composition may be obtained from any suitable source. Forskolin may be extracted from the Coleus forskohli plant, or synthetically produced. Preferably, the forskolin extract is derived from the Coleus forskohli plant and is standardized to contain about 40% forskolin. In one embodiment, forskolin comprises about 0.05-50% of the dry weight of the fertility enhancement composition, such as about 0.1-30%, or about 0.5-10% of the dry weight of the composition. In another embodiment, the fertility enhancement composition comprises a daily dosage of about 1 mg-200 mg of forskolin and preferably, about 15 mg-100 mg, for example, 15-50 mg/day, or 25 mg/day.

The fertility enhancement composition comprises at least one mitochondria-enhancing agent. The mitochondria-enhancing agent may be selected from any suitable agent which enhances mitochondrial capacity in a mammal, for example, by increasing the abundance of mitochondria, increasing the ATP generating capacity of mitochondria, protecting mitochondria from excessive oxidative stress, promoting the maintenance of mitochondrial components, ion gradients and ultrastructure, such as through increased mitochondrial autophagy (mitophagy) events or increased mitochondrial fission and fusion events. The current FE composition has been found surprisingly to improve mitochondrial function as reflected by lower rates of gonadal tissue apoptosis and lower rates of systemic and ovarian specific inflammasome-mediated inflammation. Thus, the mitochondrial agent may be any suitable agent that leads to a reduction in apoptosis and/or lower inflammation sytemically or in either testes or ovaries, in a mammal, e.g. by at least about 1%, and preferably a reduction of about 5% or more, e.g. by 10%, 15%, 20%, or greater as compared to apoptosis or inflammation prior to treatment.

In one embodiment, the mitochondria-enhancing agent is selected from at least one of the following: beetroot extract, alpha lipoic acid, nitrates, nicotinamide riboside, vitamin C, vitamin D, vitamin E, thiamine, riboflavin, magnesium, calcium, phosphate, membrane phospholipids, creatine, pyruvate, coenzyme Q10, NADH, nicotinic acid, curcumin, resveratrol and mixtures thereof.

In one embodiment, the mitochondria-enhancing agent comprises a mixture of beetroot extract, coenzyme Q10, alpha lipoic acid and vitamin E.

In another embodiment, the mitochondria-enhancing agent comprises a mixture of beetroot extract, coenzyme Q10, alpha lipoic acid, creatine and vitamin E.

The beetroot extract for use in the present composition may be selected from any suitable beetroot source including red beets such as Detroit Dark Red, Red Ace, Early Wonder Tall Top, Bull's Blood, Forono, Ruby Queen, Chioggia, Cylindra or Gladiator, yellow or gold beets such as Yellow Detroit, Golden, Touchstone Gold or Boldor or white beets such as Avalanche, Baby White, Blankoma or Sugar. Preferably, the beetroot extract is substantially derived from the taproot portion of the beetroot. In one embodiment, the beetroot extract for use in the present composition comprises at least about 1.5% nitrates by dry weight. In another embodiment, the beetroot extract comprises about 0.1-90% of the dry weight of the fertility enhancement composition, such as about 1-50%, or about 5-25% of the dry weight of the composition. In a further embodiment, the fertility enhancement composition comprises a daily dosage of about 10 mg-50 g of beetroot extract and preferably, about 50-5000 mg, for example, 50-500 mg/day, 100-300 mg/day or 250 mg/day.

Coenzyme Q10, also known as ubiquinone, ubidecarenone, coenzyme Q, CoQ10, CoQ, or Q10, may assume any one of three redox states for use in the present composition, namely, fully oxidized (ubiquinone), semi-oxidized (semiquinone or ubisemiquinone), and fully reduced (ubiquinol) forms, along with oxidized mitochondrially targeted forms of this enzyme (e.g. mitoquinone mesylate (MitoQ10)). As would be appreciated by one of skill in the art, coenzyme Q10 may be formulated in numerous ways to improve its bioavailability or effectiveness. Examples of such formulations for use in the present composition, which are not intended to be limiting, include the following: colloidal-based, solid dispersion-based, oily dispersion-based, micelle-based, nanoliposome-based, nanostructured lipid carrier-based, nanocrystal-based, nanoparticle-based, self-nanoemulsifiable-based, ascorbic acid with chelation-based and cyclodextrin complexation-based. In one embodiment, coenzyme Q10 comprises about 0.1-80% of the dry weight of the fertility enhancement composition, such as about 1-50%, or about 5-20% of the dry weight of the composition. In a further embodiment, the fertility enhancement composition comprises a daily dosage of about 10 mg-1 g of coenzyme Q10 and preferably, about 50-900 mg, for example, 100-500 mg/day or 200 mg/day.

Alpha lipoic acid suitable for use in the present composition may include, without limitation, alpha lipoic acid or its reduced form, dihydrolipoic acid, with R- and S-enantiomers either present individually, in racemic form or in any other mixture thereof. The R-enantiomer is produced naturally or synthetically, while the S-enantiomer is only produced synthetically and does not occur naturally. Additionally, any pharmaceutically acceptable salts or derivatives thereof are suitable for use in the present composition. Preferably, the alpha lipoic acid is present in racemic form. In one embodiment, the alpha lipoic acid comprises about 0.1-90% of the dry weight of the fertility enhancement composition, such as about 5-50%, or about 10-30% of the dry weight of the composition. In another embodiment, the fertility enhancement composition comprises a daily dosage of about 10 mg-10 g of alpha lipoic acid and preferably, about 50 mg-900 mg, for example, 100-500 mg/day, 100-300 mg/day or 200 mg/day.

Creatine for use in the present composition may be in any suitable form, such as creatine monohydrate, creatine anhydrous, creatine citrate, creatine ascorbate, creatine ethyl ester, creatine nitrate, creatine magnesium chelate, creatine hydrochloride, creatine malate, creatine pyruvate, creatine phosphate, creatine citrate malate, creatine tartrate, effervescent creatine, creatine titrate, buffered creatine, micronized creatine and any combination thereof. Preferably, the creatine is creatine monohydrate. In one embodiment, creatine comprises about 1%-90% of the dry weight of the fertility enhancement composition, such as about 20-70%, or about 30-50% of the dry weight of the composition. In another embodiment, the fertility enhancement composition comprises a daily dosage of about 0.1-10 g of creatine and preferably, about 0.5-5 g, for example, 3 g/day.

Vitamin E for use in the present composition may be in the form of any one or more of the isomers thereof, including alpha-tocopherol, beta-tocopherol, gamma-tocopherol, delta-tocopherol, alpha-tocotrienol, beta-tocotrienol, gamma-tocotrienol, and delta-tocotrienol, and stereoisomers thereof. Vitamin E may also be used in analogue form, including, for example, vitamin E esters (such as acetate, succinate or palmitate forms) or other forms of vitamin E which have been modified for improved stability or bioavailability. Preferably, the form of vitamin E used in the composition is alpha-tocopherol comprising biologically functional stereoisomers of alpha-tocopherol such as the naturally occurring RRR-configuration or the synthetically produced 2R-stereoisomer forms (RSR-, RRS-, and RSS-). In the most preferred embodiment, the vitamin E used is D (also known as RRR)-alpha tocopheryl acetate. In one embodiment, the vitamin E comprises about 0.1-80% of the dry weight of the fertility enhancement composition, such as about 1-50%, or about 3-15% of the dry weight of the composition. In a further embodiment, the fertility enhancement composition comprises a daily dosage of about 10 mg-1 g of vitamin E and preferably, about 50-900 IU/day, for example, 50-500 IU/day, 100-300 IU/day or 200 IU/day.

The fertility enhancement (FE) composition comprises at least one fertility-support (FS) agent. A fertility-support (FS) agent as used herein is an agent which functions to enhance fertility by maintaining or attaining healthy folate and/or iron levels, reducing systemic and/or sex organ/gonadal specific inflammation and apoptosis levels, increasing sperm function (volume, motility, count, concentration, morphology and DNA integrity), increasing oocyte and embryo quality, and/or increasing pregnancy and fertilization rate, thereby, improving successful copulation and viable offspring. Accordingly, fertility support agents may be selected to lower the risk of folate deficiency and resultant neural tube defects and to improve fertility outcomes. Such agents include, but are not limited to, agents such as; folic acid, folate salts, and folinic acid. Fertility agents may also improve the omega-3 fatty acid/docosahacanoic (DHA) status of the mother and developing fetus, and thus, may include, for example; fish oil and other sources of omega-3 fatty acids, including eicosapentaenoic acid (EPA), docosahexaenoic acid (DHA), alpha-linolenic acid (ALA), stearidonic acid, docosapentaenoic acid, and combinations thereof. The n-3 fatty acid may also be obtained from algae, dark leafy green vegetables, plant and plant seed oils. Preferred omega-3 fatty acids are DHA and/or EPA. Fertility agents may be selected to increase the iron status of the mother and developing fetus, and thus, may include, for example, ferrous gluconate, ferrous citrate, ferrous sulfate, ferrous bisglycinate, and ferrous fumarate. Fertility agents may be selected to improve the blood supply to the male sex organ/gonadals and thus may include vasodilating agents, for example, L-arginine and/or L-citrulline.

In one embodiment the fertility enhancement composition comprises as the fertility support agents, omega-3 fatty acids such as DHA and/or EPA, elemental iron, L-arginine and folate/folic acid.

In one embodiment, the fertility enhancement (FE) composition comprises as the weight loss agents, a mixture comprising a daily dosage of 25-1000 mg of green tea extract, 50-1000 mg of green coffee bean extract, 15-100 mg of forskolin, and as the mitochondria-enhancing agents, a mixture comprising a daily dosage of 50-5000 mg of beetroot extract, 50-900 mg of coenzyme Q10, 50-900 mg alpha lipoic acid, 1-5 g of creatine monohydrate, and 50-900 IU of vitamin E, and as the fertility support agents, a mixture of 200-1500 mg each of DHA and EPA as omega-3 fatty acids/fish oil, 1-100 mg of elemental iron as an iron salt, 50-2000 mg of L-arginine, and 200-1000 μg of folate/folic acid.

In embodiments, the FE composition may comprise weight loss agents in amounts as follow: green tea extract in amounts of 50-500 mg/day, 100-300 mg/day or 250 mg/day, green coffee bean extract in amounts of 50-500 mg/day, 100-300 mg/day or 250 mg/day, forskolin in amounts of 15-50 mg/day, or 25 mg/day, and mitochondria-enhancing agents in amounts as follow: beetroot extract in amounts of 50-500 mg/day, 100-300 mg/day or 250 mg/day, coenzyme Q10 in amounts of 100-500 mg/day or 200 mg/day, alpha lipoic acid in amounts of 100-500 mg/day, 100-300 mg/day or 200 mg/day, creatine monohydrate in an amount of 1-5 g/day or about 3 g/day, and vitamin E in amounts of 50-500 IU/day, 100-300 IU/day or 200 IU/day.

In embodiments, the FE composition may comprise DHA and/or EPA in amounts of about 800-1200 mg/day, 900-1100 mg/day or 950 mg/day; folic acid in amounts of about 400-800 mg/day, 500-700 mg/day or 600 mg/day); elemental iron in amounts of about 1-100 mg/day, such as 1-5 or 1-2 (e.g. 1.4) mg/day for men, or 10-80, 20-50, or 30 mg/day for women, which may in the form of ferrous gluconate or ferrous bisglycinate, and L-arginine in amounts of about 50-1000 mg/day, 150-650 mg/day, 200-300 mg/day or 210 mg/day.

In one embodiment, caffeine is present in the fertility enhancement composition within a naturally occurring source such as green tea or green coffee beans, or as anhydrous caffeine. The anhydrous caffeine may be derived from any suitable source, such as from any one of about 60 plant species naturally containing caffeine, which include tea leaves, coffee beans, cocoa beans, yerba mate, guarana berries, guayusa, and the yaupon holly. Preferably, the fertility enhancement composition comprises a daily dose of no more than about 300 mg of caffeine, such as about 50-250 mg in a caffeine-containing formulation.

In another embodiment, the fertility enhancement (FE) composition is substantially caffeine-free. In the substantially caffeine-free fertility enhancement composition, ingredients which naturally contain caffeine may be provided in decaffeinated form, such that each decaffeinated ingredient contains less than 1% of caffeine by dry weight for example. Methods for decaffeination are known in the art.

The fertility enhancement (FE) composition may comprise any food-grade source of vitamins and minerals which are suitable for oral administration to an individual. Suitable vitamins include the following non-limiting examples: vitamin A, vitamin B12 (cobalmin), vitamin C, vitamin D, vitamin K, thiamine, riboflavin, niacin, pantothenic acid, pyridoxine, folic acid/folate, biotin, carotenoids (e.g. lutein, beta-carotene, lycopene and cryptoxanthin), choline, inositol and combinations thereof, and suitable minerals include, but are not limited to, calcium, phosphorus, selenium, chromium, zinc, molybdenum, iodine, chloride, phosphorus, manganese, fluoride, potassium, iron, copper, magnesium, sodium and combinations thereof. Vitamins may be included in an amount that complies with recommended daily dosages, either alone or in combination with diet. Vitamins and minerals may be included in the composition in an amount that does not adversely affect the function of the composition. Preferred vitamins and minerals for use in the fertility enhancement composition include; vitamin D, vitamin B12, folate, copper, zinc and selenium.

The fertility enhancement (FE) composition may comprise any food-grade source of antioxidants which are suitable for oral administration to an individual. Suitable antioxidants include the following non-limiting examples: citric acid monohydrate, vitamin A, vitamin C, folic acid/folate, beta-carotene, iron, selenium, copper, butylated hydroxyamisole, butylated hydroxytoluene, propyl gallate, tertiary butylhydroquinone, resveratrol, and plant phytonutrients or phytochemicals (e.g. flavonoids and lignin). Herbs or herbal extracts (e.g. oregano, Goji berry, dill, garden thyme, rosemary and peppermint), tea leaves or tea leaf extracts (e.g. Camellia sinensis), coffee bean extracts (e.g. Coffea canephora and Coffea arabica), brewed coffee or tea or brewed coffee or tea extracts (e.g. oolong tea and Coffea robusta) and other plants or plant extracts (e.g. ginger root) may also be used as a source of antioxidants, as well as combinations of any of the antioxidants. Antioxidants may be included in the composition in an amount that does not adversely affect the function of the composition.

In one embodiment, the FE composition comprises as additional components iron, L-arginine, vitamin D, vitamin B12, zinc, copper and selenium.

Thus, in a further embodiment, the fertility enhancement (FE) composition comprises as the weight loss agents, a mixture comprising a daily dosage of 25-1000 mg of green tea extract (e.g. 50-500 mg/day, 100-300 mg/day or 250 mg/day), 50-1000 mg of green coffee bean extract (e.g. 50-500 mg/day, 100-300 mg/day or 250 mg/day), 15-100 mg of forskolin (e.g. 15-50 mg/day, or 25 mg/day), and as the mitochondria-enhancing agents, a mixture comprising a daily dosage of 50-5000 mg of beetroot extract (e.g. 50-500 mg/day, 100-300 mg/day or 250 mg/day), 50-900 mg of coenzyme Q10 (e.g. 100-500 mg/day or 200 mg/day), 50-900 mg alpha lipoic acid (e.g. 100-500 mg/day, 100-300 mg/day or 200 mg/day), 1-5 g (e.g. 3 g/day) of creatine monohydrate, and 50-900 IU of vitamin E (e.g. 50-500 IU/day, 100-300 IU/day or 200 IU/day), and as the fertility support agents, a mixture of 200-1500 mg of each of DHA and EPA, or of DHA or EPA (e.g. 400-1200 mg/day, 600-1100 mg/day or 950 mg/day), 200-1000 μg of folate/folic acid (e.g. 400-800 mg/day, 500-700 mg/day or 600 mg/day), elemental iron in amounts of about 1-100 mg/day, such as 1-5 or 1-2 (e.g. 1.4) mg/day for men, or 10-80, 20-50, or 30 mg/day for women, which may in the form of ferrous gluconate or ferrous bisglycinate, and L-arginine in amounts of about 50-1000 mg·day, 150-650 mg/day, 200-300 mg/day or 210 mg/day, as well as vitamin D (400-2,000 IU/day, e.g. 500-1500 IU/day, or 1,000 IU/day), vitamin B12 (10-100 μg/day, e.g. 20-80 μg/day, 40-60 μg/day or 50 μg/day), zinc (10-50 mg/day, e.g. 20-40 mg/day or 30 mg/day), copper (1-5 mg/day, e.g. 3 mg/day), and selenium (10-125 ug/day, e.g. 30-100 μg/day, 50-80 μg/day or 70 μg/day).

The present FE composition may additionally include at least one physiologically acceptable excipient. The term “physiologically acceptable” is used herein to refer to excipients which are food-grade and thus, acceptable for consumption or administration to a mammal. Examples of suitable excipients, which are not to be construed as limiting, include flavouring agents, sweetening agents, anti-caking agents/flowing agents, emulsifiers, stabilizers, masking agents, colorants, preservatives, disintegrants, binders, thickeners and pH adjusters.

Non-limiting examples of flavouring agents include natural or artificial flavours such as fruit flavours (e.g. raspberry, orange, apple, pomegranate, mixed berry, lemon, lime, watermelon, strawberry, blueberry, pineapple, coconut, grape, cherry, banana, peach, mango, kiwifruit, cranberry), sodium sources (e.g. sodium chloride and monosodium glutamate), high fructose corn syrup, vanilla, chocolate, unsweetened chocolate, honey, molasses, brown sugar, coffee, cocoa, mint, maple, almond, or extracts or combinations thereof. Savoury flavourings may also be used (e.g. beef, chicken or vegetable flavourings).

Non-limiting examples of sweetening agents include natural sweeteners such as, glucose, fructose, sucrose, dextrose, maltose, brown sugar, molasses, honey, maple syrup, corn syrup, high fructose corn syrup, erythritol, xylitol, sorbitol, isomalt, monatin, monellin, curculin, brazzein, tagatose and mannitol, and artificial sweeteners such as aspartame, acesulfame K, saccharin cyclamate and sucralose.

Non-limiting examples of further excipients include: anti-caking agents or flowing agents such as silicates (e.g. silicon dioxide) and calcium or magnesium stearates; emulsifiers such as agar, gums, egg yok, lecithin, monostearate, monosodium phosphate, monoglycerides, diglycerides and alginates; stabilizers such as glycerine, agar, gums, alginates and pectin; masking agents such as glycerine, sodium chloride, peppermint, lemon-lime, mint, cherry, black liquorice, peach, apricot, raspberry, or sweetening agents such as aspartame or sucrose; colorants such as those which are suitable for inclusion in foods, e.g. FD&C blue #1, FD&C blue #2, FD&C citrus red #2, FD&C green #3, FD&C red #3, FD&C red #40, FD&C yellow #5 and FD&C yellow #6; preservatives such as butylated hydroxyanisole, butylated hydroxytoluene, ethylenediaminetetraacetic acid, nitrates (e.g. sodium nitrate), sulfites (sodium bisulfite), benzoates (sodium benzoate), sorbates (e.g. sodium sorbate) and sodium chloride; disintegrants such as starches (e.g. potato starch), alginic acid, cellulose and derivatives thereof, and calcium silicate; binders such as stearic acid, gelatin, saccharides and derivatives thereof, sugar alcohols, polyethylene glycol and cellulose; thickeners such as polysaccharide-based thickeners such as vegetable gums, pectin and starches or protein-based thickeners such as gelatin, egg white and collagen; and pH adjusters such as citric acid, ammonium carbonate, ammonium phosphate, calcium carbonate, sodium hydroxide, malic acid and phosphoric acid. As will be appreciated by one of skill in the art, for each type of excipient (e.g. flavouring agent, sweetener, emulsifier, preservative, etc.), a single excipient may be used, or a combination of two or more may be used.

The fertility enhancement (FE) composition may be formulated for oral administration including, for example, solid, semi-solid, liquid, semi-liquid, powder, suspension, emulsion, solution, ready-to-drink beverage, gum, gel, bar, pill, tablet or capsule form. The term “oral” or “orally” as used herein is intended to include any method in which the fertility enhancement composition is introduced into the digestive tract including the stomach and small intestine. Examples of oral administration may include administration via mouth, directly into the stomach using a feeding tube, through the nose to the stomach via a feeding tube and through the nose to the small intestine via a feeding tube. In a preferred embodiment, the fertility enhancement composition is provided as a loose powder, a bar or in capsules. The loose powdered composition may be reconstituted in water or any suitable liquid (such as juice, milk, saline, etc.) immediately prior to consumption. When provided in loose powdered form, the fertility enhancement composition may be packaged in individual use containers, packets or sachets, or in larger bulk containers. When provided as a powder in capsules, any suitable capsule may be used including gelatin and hard hydroxypropyl methylcellulose (also known as Hypromellose) capsules.

The fertility enhancement (FE) composition may be administered in a daily effective amount to a mammal in need thereof, one or more times per day, for a period ranging from one day to chronic or long-term administration. The term “mammal” is used herein to refer to human and non-human mammals such as domestic animals (cats, dogs, horses and other livestock). The term “daily effective amount” as used herein, refers to an amount which achieves the effects desired in the mammal, without surpassing any amount which may cause undesirable side effects. For example, a daily effective amount of the fertility enhancement composition may be administered once per day, or a daily effective amount of the fertility enhancement composition may be divided into 2, 3, 4, 5, 6 or more portions to be administered throughout the day. In one embodiment, the fertility enhancement composition is administered once a day to an individual in the morning when first waking up. The fertility enhancement composition may be administered to an individual in need thereof for 1, 2, 3, 4, 5, 6 or 7 days in a week and for 1, 2, 3, 4, 5, 6, 7, 8, 9, 10 or more weeks. In one embodiment, the fertility enhancement composition is administered chronically to an individual in need thereof. The term “chronically” as used herein refers to the administration of the fertility enhancement composition for a period of at least 2-4 or more months, for example, administration of the fertility enhancement composition on a continual basis beyond 6 months, at a frequency of at least 2 days/week, and preferably at least 3 or more days a week. In one embodiment, the daily effective amount of the fertility enhancement composition is consumed over two servings, with one serving being administered in the morning and the remaining serving being administered in the afternoon or evening for a period of 3 months. In another embodiment, the daily effective amount of the fertility enhancement composition is consumed over three servings, with one serving being administered in the morning, the second serving being administered around midday and the third serving being administered in the evening for a period of 3 months.

The components of the FE composition may be administered in conjunction, either together, in a single composition, or individually in separate dosage forms which may be the same or different, and which may be administered at the same time or at different times. For example, the weight loss agents may be administered in tablet form, while the mitochondrial-enhancing agents and/or fertility-support agents may be administered separately in a different administrable dosage form, such as a capsule. The mitochondrial agents and/or the fertility-support agents may be administered at the same time as the weight loss agents are administered, or at a different time, and at the same frequency or at a different frequency. For example, the weight loss tablets may be administered twice daily, while the mitochondrial-enhancing and fertility-support capsules are administered once or twice a day. In one embodiment, each of the fertility enhancement composition components are provided in capsule form, together or separately, with the exception of creatine, which is provided in tablet or powder form.

In a further aspect of the invention, a kit is provided comprising two or more separate components of the FE composition. The first component comprises a first composition that may be in the form of a powder provided in bulk form or as individual doses, e.g. in packets, sachets or capsules, and the second component comprises a second composition that may be in the form of an oil in bulk form or as individual doses, e.g. in capsules.

In one embodiment, the second composition comprises omega-3 fatty acids such as EPA, DHA, ALA, stearidonic acid, docosapentaenoic acid, and/or combinations thereof. Preferably, the n-3 fatty acid is a combination of EPA and DHA. The amount of the selected omega-3 fatty acid(s) in the second composition is as specified herein. The first composition comprises the other components of the FE composition including the weight loss agents, the mitochondria-enhancing agents and the remaining fertility support agents. For example, the first composition comprises green tea extract, green coffee bean extract, forskolin, beetroot extract, coenzyme Q10, alpha lipoic acid, creatine monohydrate, vitamin E, folate/folic acid, iron and L-arginine in amounts as specified herein.

In another embodiment, the first composition comprises the weight loss agents, the second composition comprises the mitochondria-enhancing agents and a third composition comprises the fertility support agents, examples of each of which are described above.

In a further embodiment, compositions within the kit may additionally include one or more of the components iron, L-arginine, vitamin D, vitamin B12, zinc, copper and selenium, or one composition within the kit may include all of these components. Alternatively, these components may be included in the kit in a separate composition.

Each of the compositions of the kit may additionally include at least one physiological acceptable excipient, e.g. an excipient that does not impact the function of the compositions. Examples of suitable excipients are known in the art, and include, for example, flavouring agents, sweetening agents, anti-caking agents, flowing agents, emulsifiers, stabilizers, masking agents, colorants, preservatives, disintegrants, binders, thickeners, pH adjusters, carbohydrate and oil.

The present fertility enhancement (FE) composition is useful in a method to treat or improve at least one of the following in a mammal: infertility, body weight, body fat, body composition index, mitochondrial dysfunction, oxidative stress levels, folate or iron deficiency, and systemic or sex organ/gonadal specific inflammation and apoptosis levels. The term “individual” is used herein to refer to a mammal, including both human and non-human mammals such as dogs, cats, horses, pigs, sheep, goats, cows, rodents and the like. Preferably, the mammal is a human. Any individual may be treated using the present method, including individuals of any reproductive age.

The present treatment method comprises administering to the individual an effective amount of the fertility enhancement composition. The terms “treat”, “treating” or “treatment” are used herein to refer to methods that favourably alter: infertility, body weight, body fat, body composition index, mitochondrial energy generating capacity, oxidative stress levels, folate or iron deficiency, systemic or gonadal tissue inflammation levels, relative omega-3 deficiency, apoptotic seminiferous tubules or ovarian follicles and/or mitochondrial energy generating capacity of the sex organs, including those that moderate, reverse, reduce the severity of, or protect against infertility. The term “improve” is used herein with respect to the variables of body weight, body fat, body composition index, mitochondrial capacity, oxidative stress levels, folate or iron deficiency, omega-3 levels, apoptosis of gonadal tissue, and systemic or sex organ/gonadal specific inflammation levels refer to either a healthy increase or a healthy decrease in the variable, i.e. an increase or decrease in the variable, whichever is considered to promote health. For example, generally the treatment will achieve at least one of; weight/body fat loss, an increase in mitochondrial capacity, an increase in body composition index, an increase in omega-3 concentration, fewer apoptotic cells within gonadal tissue, a decrease in systemic or sex organ/gonadal inflammation, and/or a decrease in oxidative stress, that ultimately lead to; an increase in viable follicles, a decrease in apoptotic follicles, a decrease in apoptotic seminiferous tubules, and an increase in successful copulation and viable pregnancies.

The present method is useful for individuals, either male or female individuals, that require fertility enhancement. Administration of the present fertility enhance composition is effective to enhance fertility by treatment of one or more of, excess body weight (overweight), excess body fat (obese), sarcopenic obesity (low body composition index), mitochondrial dysfunction and/or an increase in oxidative stress (e.g. aging, primary mitochondrial disease, etc.) and/or an increase in systemic and/or gonadal inflammation (e.g. aging, autoimmune diseases, etc.), or idiopathic factors (i.e. infertility in otherwise healthy younger individuals, including males and females, e.g. individuals who are 30 years old or less or the equivalent. Older aged individuals for the purposes of fertility are considered herein to be greater than 30 years of age, or the equivalent).

As used herein, the term “body weight” refers to the total body mass of an individual or to the mass of specific regions of the body. Improved body weight is a reduction of body weight in an individual by at least about 0.5%, and preferably a reduction of about 1% or more, e.g. by 5%, 10%, 30%, 50%, 70% or greater of body weight in the individual prior to treatment with the present composition or a reduction in the rate of rising body weight by at least about 1% or more, and preferably a reduction of about 5% or more, e.g. by 10%, 30%, 50%, 70%, 90% or greater to the rate of body weight increase prior to treatment. With respect to body weight, the present composition surprisingly exhibits a synergistic effect, i.e. the effect of the combination of a weight loss agent and a mitochondrial-enhancing agent results in a reduction of body weight that is greater than the additive effect of a weight loss agent alone, and a mitochondrial-enhancing agent alone. This in turn will be reflected in other treatments for which the composition is useful insofar as an improvement in body weight and reduction in body fat promotes such treatments, e.g. treatment of subfertility.

As used herein, the term “body fat” refers to the total fat mass of an individual or to the fat mass of specific regions of the body. During healthy weight loss, the majority of body weight loss is derived from the reduction of excess fat stores in an individual. Improved body fat is a reduction of body fat in an individual by at least about 0.5%, and preferably a reduction of about 1% or more, e.g. by 5%, 10%, 30%, 50%, 70% or greater of body fat in the individual prior to treatment with the present composition, or a reduction in the rate of rising body fat by at least about 1% or more, and preferably a reduction of about 5% or more, e.g. by 10%, 30%, 50%, 70%, 90% or greater to the rate of body fat increase prior to treatment.

As used herein, the term “body composition index” (BCI) refers to the total lean body mass of an individual divided by the total fat mass of the body. These may be determined using MRI spectroscopy, direct measurement or dual-energy X-Ray absorptiometry (DEXA), bioelectrical impedance (BIA), air displacement (BOD-POD), or hydrostatic weight methods in humans. Improved BCI is an increase of BCI in an individual by at least about 0.5%, and preferably an increase of about 1% or more, e.g. by 5%, 10%, 30%, 50%, 70% or greater of BCI in the individual prior to treatment with the present composition or a reduction in the rate of rising body weight by at least about 1% or more, and preferably a reduction of about 5% or more, e.g. by 10%, 30%, 50%, 70%, 90% or greater to the rate of body weight increase prior to treatment. With respect to body weight, the present composition surprisingly exhibits a synergistic effect, i.e. the effect of the combination of weight loss agents and mitochondrial-enhancing agents results in a reduction of body weight that is greater than the additive effect of weight loss agents alone, and mitochondrial-enhancing agents alone. This in turn will be reflected in other treatments for which the composition is useful insofar as an improvement in body weight and reduction in body fat promotes such treatments, e.g. treatment of infertility.

As used herein, the term “mitochondrial capacity” refers to the total physiological productive capacity for mitochondria to produce cellular energy stores by carrying out cellular respiration. The term encompasses the mitochondrial capacity of one or more mitochondria and may be used to refer to the mitochondrial capacity of mitochondria within specific regions (such as specific muscles or organs) or within the entire body. Important indicators of mitochondrial capacity include the following non-limiting examples: an increase in the mRNA expression of mitochondrial capacity biomarkers such as PGC1 alpha, COX2, PPAR alpha, HSL, SCHAD, UCP3, PRDM16, CIDEA and AIPOQ, an increase in the total number of mitochondria (also known as mitochondrial volume density) in a tissue, an increase in the enzymatic capacity expressed in absolute terms or relative to the total number of mitochondria measured, the presence of biomarkers of mitochondrial integrity, such as the absence of deletions, cristae fragmentation or pleomorphism and a heightened amount (but not an excessive amount) of mitochondrial turnover via mitophagy or mitochondrial fission and fusion events. For example, improved mitochondrial capacity refers to an increase in the mRNA expression of mitochondrial capacity biomarkers in an individual by at least about 0.5%, and preferably an increase of about 1% or more, e.g. by 5%, 10%, 30%, 50%, 70% or greater from the mRNA expression of mitochondrial capacity biomarkers of the individual prior to treatment with the present composition, or at least a reduction in the rate of mRNA expression of mitochondrial capacity biomarker decline in an individual who is experiencing a decrease in the mRNA expression of mitochondrial capacity biomarkers by at least about 1% or more, and preferably a reduction in the rate of mitochondrial capacity loss by about 5% or more, e.g. by 10%, 30%, 50%, 70%, 90% or greater from the rate of mRNA expression of mitochondrial capacity biomarker decline in the individual prior to treatment with the present composition. Improved mitochondrial capacity may also refer to a normalization of mitochondrial capacity in one or more tissues within the levels of a healthy individual.

As used herein, the term “systemic inflammation level” refers to the amount of inflammation (including inflammasome activation) present in circulation (protein) and within tissues (protein or mRNA) of the body and/or in specific tissues (ovaries or testes). While acute inflammation in response to injurious events is thought to be beneficial, the presence of a chronic-low grade inflammation level is generally thought to be deleterious and likely related to the development of other chronic diseases associated with obesity. Systemic inflammation level is most commonly evaluated by measuring the mRNA expression or protein levels of pro-inflammatory biomarkers such as C-reactive protein (CRP), interleukin-6 (IL-6), IL-1beta (IL-1B) and tumor necrosis factor-alpha (TNF-α) in circulation, and markers of inflammasome (NLRP3) activation in tissues (CASP1, IL-1B, and IL-18). Other common biomarkers of systemic inflammation include pro-inflammatory cytokines such as IL-1α, IL-12 and IL-18, chemokines such as CXCL-8, CCL2, CCL3, CCL4, CCL5, CCL11 and CCXCL10 and growth factors such as GM-CSF, PDGF, TGF-Beta and VEGF. A change in levels of anti-inflammatory cytokines, such as IL-2, IL-4, Il-10 and IL-13, may also accompany the pro-inflammatory state. An improved systemic inflammation level is a reduction in the mRNA expression of pro-inflammation biomarkers in an individual and/or gonadal tissue by at least about 0.5%, and preferably a reduction of about 1% or more, e.g. by 5%, 10%, 30%, 50%, 70% or greater to the mRNA expression of inflammation biomarkers prior to treatment with the present composition, or at least a reduction in the rate of an increase of mRNA expression of inflammation biomarkers by at least about 1% or more, and preferably a reduction of the rate of increase of mRNA expression of inflammation biomarkers by about 5% or more, e.g. by 10%, 30%, 50%, 70%, 90% or greater to the rate of mRNA expression of inflammation biomarkers prior to treatment with the present composition.

As used herein, the term “oxidative stress” refers to an imbalance between production and accumulation of oxygen reactive species (ROS) in cells and tissues and the ability of a biological system to detoxify these reactive products. Biomarkers of oxidative stress include products of lipid peroxidation, isoprostanes (IsoPs), isomers of prostaglandins, for example, 8-iso-prostaglandin F_{2α} (8-iso-PGF_{2α}), as well as free radicals such as malondialdehyde (MDA) and trans-4-hydroxy-2-nonenal (4-HNE), and reactive oxygen species (ROS). An improved level of oxidative stress is a reduction in the level of one or more of these biomarkers in an individual and/or gonadal tissue by at least about 0.5%, and preferably a reduction of about 1% or more, e.g. by 5%, 10%, 30%, 50%, 70% or greater to the levels prior to treatment with the present composition, or at least a reduction in the rate of increase of these biomarker levels by at least about 1% or more, and preferably a reduction of the rate of increase of an oxidative stress biomarker by about 5% or more, e.g. by 10%, 30%, 50%, 70%, 90% or greater to the rate of increase of such biomarker levels prior to treatment with the present composition.

As used herein, the term “reduction in apoptosis” refers to a reduction in the proportion of apoptotic cells present within a sex organ (ovary (#/ovary) or testes (% seminiferous tubules with apoptosis)). The quantification of apoptotic cells within a tissue is conducted using TUNEL staining but can also be determined using other methods for determining the activation of apoptosis such as COMET assay, caspase-3 activation, or the mRNA abundance of apoptotic factors (bax, bcl-2, caspase-3,6,8,9) within affected tissues. An improvement in apoptosis is a reduction/lowering of the proportion of cells within a relevant tissue (ovary, testes) undergoing apoptosis or a reduction in apoptosis biomarkers within gonadal tissue by at least about 0.5%, and preferably a reduction of about 1% or more, e.g. by 5%, 10%, 30%, 50%, 70% or greater as compared to such biomarkers prior to treatment with the present composition or when comparing mammals treated with the present composition vs those treated with a placebo.

The present method is also useful to treat infertility in a mammalian male and female couple. Thus, the fertility enhancement composition is, thus administered to both the male and female of the couple a fertility-enhancement composition to enhance fertility in both the male and female of the couple by treating one or more of body weight, body fat, body composition index, mitochondrial dysfunction, fatty liver disease, dyslipidemia, oxidative stress levels, gonadal cell-cycle impairment, gonadal apoptosis/atrophy, and systemic and/or gonadal inflammation levels.

Unless otherwise indicated, the definitions and embodiments described in this and other sections are intended to be applicable to all embodiments and aspects of the present application herein described for which they are suitable as would be understood by a person skilled in the art.

### EXAMPLES

### Example 1—Fertility Enhancement Composition Improves Bodyweight, Bodyfat Fertility, Gonadal Pathology and Inflammation In Vivo

To determine if a fertility enhancement (FE) composition, in accordance with an embodiment of the invention, comprising weight loss agents, mitochondria-enhancing agents, and fertility-support agents could: 1) reduce body weight and body fat (improved body composition index); and/or 2) lower inflammatory markers and histological evidence (apoptosis, cell death and atrophy) of obesity-associated ovarian and/or testicular pathology; and/or 3) improve the number of offspring arising from successful mating (copulatory success/couple fertility), in Western diet fed mice (HF), the following experiments were conducted.

Five-month-old male and female C57BL/6J mice were fed a low-fat diet (LF) or a high fat/sucrose Western diet (HF) for six weeks (obesity induction period), followed by six weeks of LF (LF), HF (HF) or HF combined with a fertility enhancement composition (FE) (intervention period). The low-fat control diet (LF) contained 3.64 kcal/g made up by protein 19% kcal, carbohydrates 68.2% kcal (sucrose 120 g/kg) and fat 12.8% kcal. The high-fat/sucrose Western diet (HF) contained 4.56 kcal/g made up by protein 15.3% kcal, carbohydrates 42.8% kcal (sucrose 345 g/kg) and fat 41.9% kcal. The high-fat, fertility enhancement composition (FE) was calorie-matched to HF and contained 4.5 kcal/g made up by protein 16.6% kcal, carbohydrates 37.1% kcal (sucrose 345 g/kg) and fat 46.2% kcal. The FE diet also contained weight loss agents (green coffee bean extract; 2.5 g/kg, green tea extract; 0.75 g/kg and forskolin; 0.125 g/kg), mitochondrial enhancers (α-lipoic acid; 1 g/kg, CoQ10; 2.5 g/kg, vitamin E/a tocopheryl acetate; 2.1875 g/kg and beetroot extract; 10 g/kg; creatine monohydrate (30 g/kg)), and fertility support agents (L-arginine (5 g/kg), iron/ferric citrate (1.21 g/kg), folic acid (0.005 g/kg), omega-3 (28 g/kg)), vitamin D3—2.4 mg/kg, vitamin B12—0.05 mg/kg, zinc—80 mg/kg, copper—25 mg/kg, and selenium—0.34 mg/kg. A sub-set of animals were sacrificed by cervical location at 12 weeks for assessment of total WAT mass, gonadal histopathology (Hematoxylin & Eosin stain), cell death (TUNEL stain) and inflammation (mRNA levels by qRT-PCR), while the remainder were harem-mated in a 1:2 male-to-female ratio for five days, and thereafter singly housed during a three-week gestational period. During mating and gestation all animals were fed the same diets that they had been fed for the previous 12 weeks. At the end of the feeding intervention all mice had their total lean body mass and fat mass determined using magnetic resonance imaging (MRI) to determine the body composition index (BCI). All mice, including pups, were euthanized by CO₂ immediately after birth of the litters. All experiments were approved by the McMaster University Animals Ethics Committee and conducted under appropriate Canadian guidelines for animal research.

### Results

To assess if the fertility enhancement composition improved bodyweight (BW), fat mass, and body composition index (BCI), BW and total white adipose tissue (WAT; perirenal, mesenteric, gonadal and inguinal) were measured following six weeks of intervention. BW and total WAT were robustly improved by the fertility enhancement composition in both sexes, and were even lower in the FE group compared to the LF group in females (Table 1). These results demonstrate that the fertility enhancement composition improves bodyweight and lowers fat mass. BCI was determined using the equation (total body lean mass/total body fat mass, w/w). Furthermore, the BCI was lower in the HF animals and dramatically improved FE animals to values similar to those in LF animals (Table 1).

TABLE 1
Effect of multi-nutrient supplementation on food consumption, bodyweights, total white
adipose tissue mass, and body composition index (BCI) in Western Diet-fed mice.

| | MALE | | | FEMALE | | |
|---|---|---|---|---|---|---|
| | LF | HF | FE | LF | HF | FE |
| | (n = 18) | (n = 15) | (n = 16) | (n = 28) | (n = 26) | (n = 26) |
| Food Intake | | | | | | |
| (all mice] | | | | | | |
| g/day | 3.4 ± 0.1 | 3.1 ± 0.1* | 2.6 ± 0.1§ | 3.0 ± 0.0 | 2.7 ± 0.1* | 2.2 ± 0.0§ |
| kcal/day | 12.3 ± 0.3 | 14.5 ± 0.3* | 11.9 ± 0.4§ | 10.6 ± 0.1 | 12.2 ± 0.3* | 10.0 ± 0.2§ |
| Body Weight | | | | | | |
| (all mice] | | | | | | |
| BW (g) | 38.0 ± 0.9 | 45.8 ± 0.9* | 39.7 ± 1.5§ | 27.1 ± 0.8 | 31.8 ± 1.0* | 24.1 ± 0.4§ |
| Fat Mass | | | | | | |
| [n = 6/group) | | | | | | |
| Total WAT (mg) | 3367 ± 376 | 6223 ± 297* | 5092 ± 560§ | 1335 ± 399 | 3186 ± 499* | 762 ± 245§ |
| Body Composition | | | | | | |
| [n = 6/group] | | | | | | |
| Body composition index | 1.93 ± 0.15 | 1.10 ± 0.04* | 1.31 ± 0.12§ | 4.05 ± 0.61 | 1.50 ± 0.19* | 5.47 ± 1.20§ |

LF = Low Fat Diet, HF = Western Diet (WD), and FE = ‘Fertility Enhancer’ multi-nutrient supplement combined with WD. Total WAT is the sum of mesenteric, inguinal, peri-renal, and gonadal deposits. The body composition index (BCI) is total lean mass divided by total fat mass assessed by MRI.
*Significantly different from LF (p ≤ 0.05).
§Significantly different from HF (p ≤ 0.05).

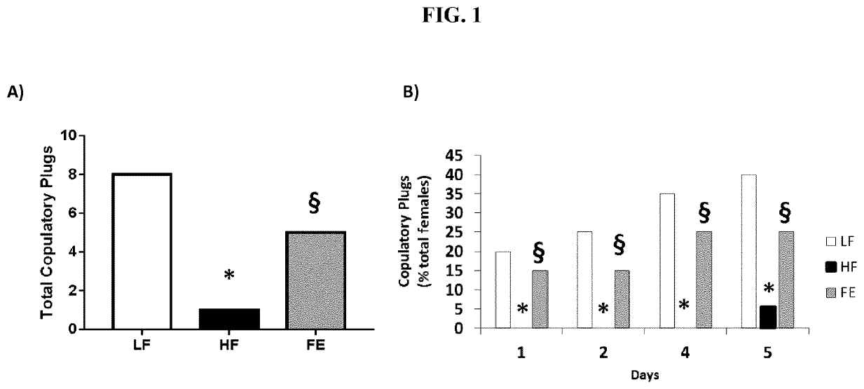

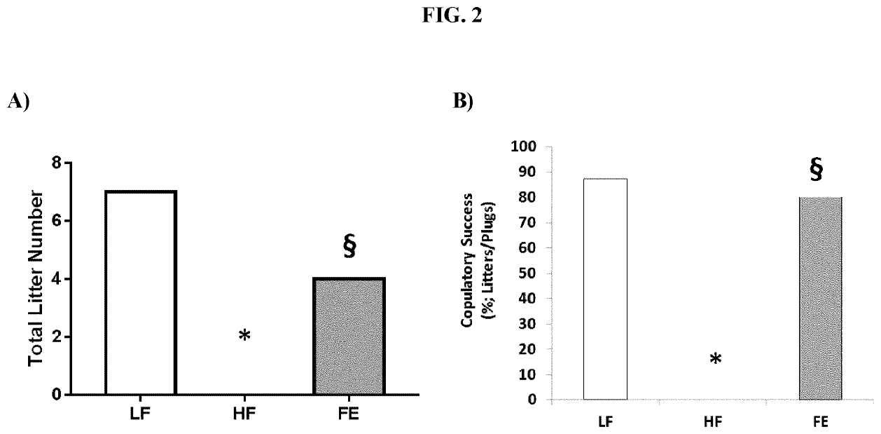

To assess whether the fertility enhancement composition enhanced couple fertility and reproductive success, sperm plugs were counted during 5 days of harem breeding and number of litters were counted following three weeks of gestation in LF, HF and FE groups. Copulatory plugs, number of litters and reproductive success were all higher in the FE group compared to the HF group and even approach the normal state (LF) (Table 2). These results are illustrated in FIGS. 1 and 2, and surprisingly show that the fertility enhancement composition improves reproductive success and couple fertility.

TABLE 2
Effect of multi-nutrient supplementation on mating behavior
and reproductive outcomes in Western Diet-fed C57BL/6J mice.

| Mating and Reproductive Outcomes | LF | HF | FE |
|---|---|---|---|
| Copulatory Plugs (5-day total) | 8 | 1* | 5§ |
| Copulatory Plugs (% total females) | 40 | 6* | 25§ |
| Number of Litters (total) | 7 | 0* | 4§ |
| Copulatory Success (%; Litters/Plugs) | 87.5 | 0* | 80§ |

Harem-breeding of mice occurred in a 1:2 male-to-female ratio during a 5-day period. Vaginal plugs were counted 6 am each morning and females singly housed following mating. LF = Low Fat Diet, HF = Western Diet (WD), and FE = ‘Fertility Enhancer’ multi-nutrient supplement with WD.
*Significantly different from LF (p ≤ 0.05).
§Significantly different from HF (p ≤0.05).

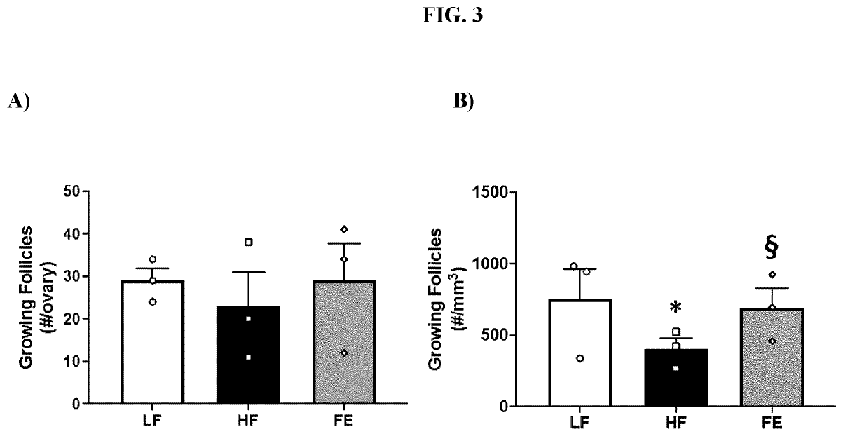

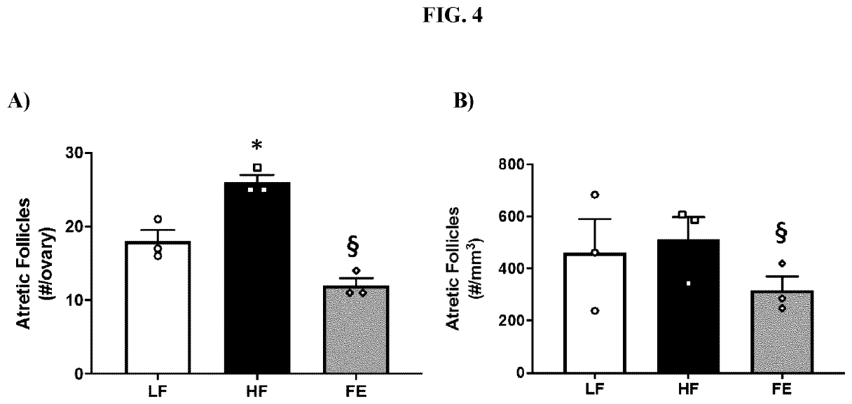

In order to assess if the fertility enhancement composition improved gonadal pathology, histopathology was done by hematoxylin & eosin and TUNEL staining in testicles and ovaries of all experimental groups. In testicles (tubules with evidence of apoptosis (%)) and ovaries (#apoptotic granulosa cells/ovary) respectively, there was less cell death (apoptosis) in the FE vs HF groups (Table 3). Furthermore, there were a higher number of growing follicles (FIG. 3) and less atresia (FIG. 4) in ovaries of FE-treated vs HF animals (Table 3), collectively showing that gonadal histopathology was improved by the fertility enhancement composition in both genders.

TABLE 3
Effect of multi-nutrient supplementation on gonadal pathology in Western Diet-fed C57BL/6J mice.

| | MALE | | | FEMALE | | |
|---|---|---|---|---|---|---|
| | LF | HF | FE | LF | HF | FE |
| Gonadal Pathology | (n = 5) | (n = 5) | (n = 5) | (n = 5) | (n = 5) | (n = 5) |
| Testicular | | | | | | |
| Seminiferous Epithelia (pathology score) | 0.9 ± 0.5 | 0.7 ± 0.2 | 0.5 ± 0.2 | NA | NA | NA |
| Tubules with Evidence of Apoptosis (%) | 29.5 ± 1.7 | 38.1 ± 17.8 | 28.6 ± 5.4 | NA | NA | NA |
| Ovarian | | | | | | |
| Viable Follicles (#/ovary) | NA | NA | NA | 61.7 ± 10.3 | 55.7 ± 19.9 | 55.7 ± 17.6 |
| Viable Follicles (#/mm³) | NA | NA | NA | 1574.4 ± 465.6 | 986.1 ± 296.5 | 1344.5 ± 301.7 |
| Growing Follicles (#/ovary) | NA | NA | NA | 29.0 ± 2.9 | 23.0 ± 7.9 | 29.0 ± 8.7 |
| Growing Follicles (#/mm³) | NA | NA | NA | 754.5 ± 209.2 | 402.9 ± 73.9* | 691.6 ± 134.6§ |
| Atretic Follicles (#/ovary) | NA | NA | NA | 18.0 ± 1.5 | 26.0 ± 1.0* | 12.0 ± 1.0§ |
| Atretic Follicles (#/mm³) | NA | NA | NA | 461.6 ± 128.5 | 512.8 ± 85.0 | 317.6 ± 52.1§ |
| Apoptotic Granulosa Cells (#/ovary) | NA | NA | NA | 424.0 ± 109.1 | 606.5 ± 321.1 | 364.2 ± 81.6 |
| Apoptotic Granulosa Cells (#/follicle) | NA | NA | NA | 15.1 ± 5.7 | 15.6 ± 7.5 | 14.5 ± 2.9 |

Seminiferous epithelia pathology and ovarian follicular stages assessed in hematoxylin and eosin-stained paraffin cross-sections (200X total magnification; Image J Software). Apoptotic cells counted in seminiferous tubules and ovarian follicles following TUNEL-staining (200X). Seminiferous epithelia were scored from 0 (healthy), 1 (mild), 2 (moderate), to 3 (severe) pathology. Viable follicles = all follicles counted excluding atretic and corpora lutea. Growing follicles = all follicles counted excluding primordial, atretic and corpora lutea. Follicles containing dark, pyknotic nuclei within the granulosa cells were considered atretic. LF = Low Fat Diet, HF = Western Diet (WD), and FE = ‘Fertility Enhancer’ multi-nutrient supplement with WD.
*Significantly different from LF (p ≤ 0.05).
§Significantly different from HF (p ≤ 0.05).

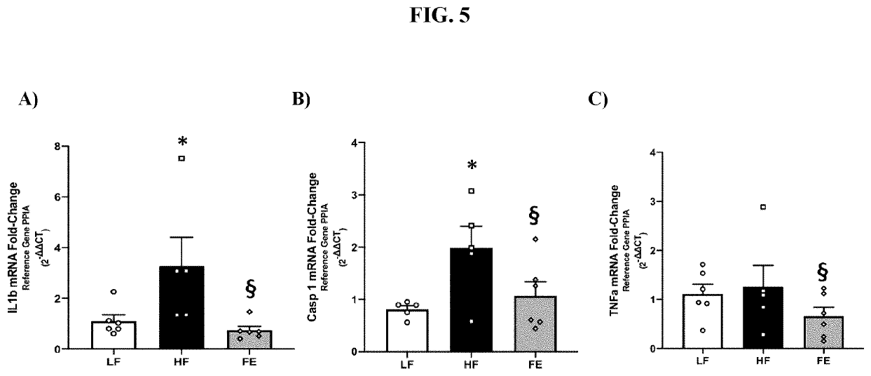

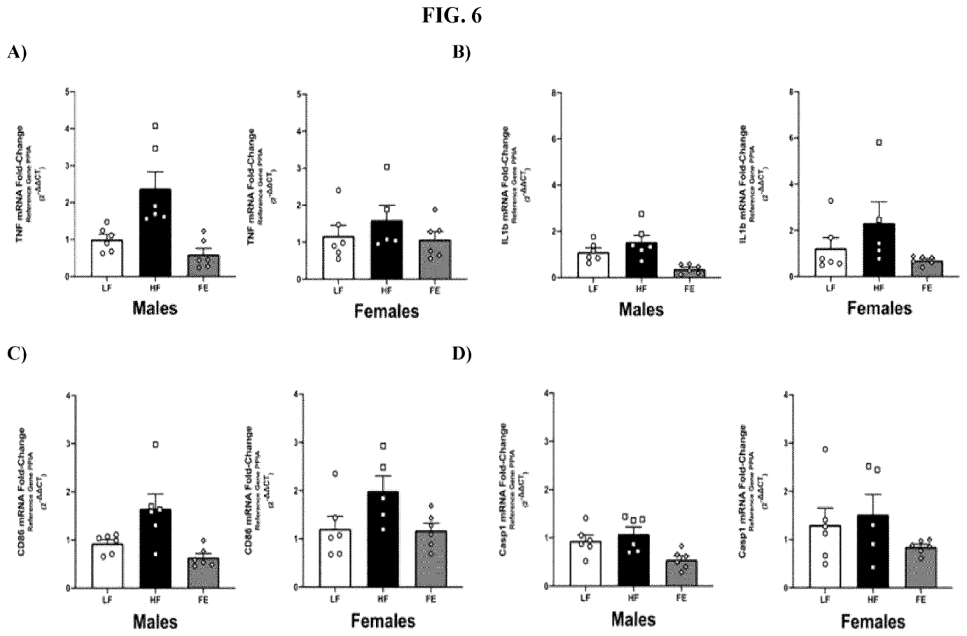

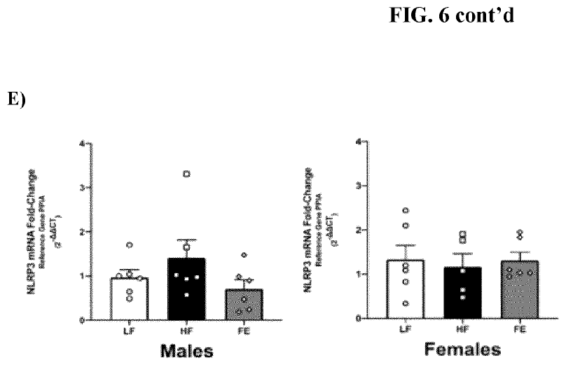

For measurement of gonadal inflammation, messenger RNA levels of key drivers of inflammation in ovaries by qRT-PCR in LF, HF and FE groups was assessed. Unexpectedly, both inflammasome (mitochondrial mediated—IL-1β, Casp 1) and non-mitochondrial mediated (TNF-α) inflammation mRNA levels were lower in ovaries from the FE-treated vs HF animals (FIG. 5). The effect of the FE on systemic markers of inflammasome mediated (IL-1β, Casp 1, NLRP3) and general mediated (CD86, TNF-α) inflammation in the liver of both male and female animals was determined (FIG. 6). Overall, this data demonstrates that major inducers of the inflammatory cascade and inflammasome activation were attenuated by the fertility enhancement composition.

In summary, these findings surprisingly show that administration of the fertility enhancement (FE) composition: 1) reduces body weight, body fat and improves BCI, 2) lowers molecular markers associated with infertility (inflammation and apoptosis) and histological evidence of obesity-associated ovarian and/or testicular pathology, and 3) improves the number of offspring arising from successful mating (copulatory success/couple fertility).

### Example 2—Fertility Enhancement Composition Improves Subfertility, Ovarian Pathology Markers and Body Composition in Younger and Older Females

To determine if a fertility enhancement (FE) composition, in accordance with an embodiment of the invention, comprising mitochondria-enhancing agents, weight loss agents and fertility-support agents could: 1) increase the number of litters arising from successful mating (copulatory success); 2) lower ovarian pathology markers (inflammation and apoptosis); and 3) reduce body weight and body fat in both younger and older mice, the following experiments were conducted.

Young (6.2 months; n=22) and old (11.7 months, n=22) female C57BL/6J mice were fed either a control (CON) or Fertility Enhancer (FE) diet for five weeks, followed by harem breeding (1:2 or 1:3 male-to-female ratio) with young males (6 months; n=16) for 5 days (one estrous cycle), followed by 21 days of gestation and litter birth. One week following the litter births, the exact same male and females underwent a second breeding period for 10 days (two estrous cycles), followed by another standard gestation period and new litter births. All breeding males were fed the control diet during this portion of the experiment, while females continued consuming their experimental diets (CON or FE) throughout the experiment. Following the last birth of litters, all female mice were sacrificed by cervical dislocation, organs harvested (ovaries, gonadal white adipose tissue, and muscle), and stored at −80° C. until analyzed for markers of pathology (oxidative damage, inflammation, and apoptosis). At the time of organ harvest, young (Y-CON and Y-FE) and old (0-CON and O-FE) mice were 9.2-month (Y) vs. 14.7-month-old (O), respectively. The primary outcomes measured for this arm of the study were sperm plugs (evidence of successful mating), number of litters, and copulatory success ([sperm plugs/litters]*100). Secondary outcomes were bodyweight and body composition, including total fat mass, muscle mass, and white adipose tissue mass. In a second arm of this study, virgin females (6.2 months old; n=4 per experimental group) underwent dietary intervention for five weeks, did not undergo breeding, and were then assessed in a metabolic system for in vivo fat oxidation rates.

The control diet (CON; TD 8604) contained 3.0 kcal/g made up by protein 32% kcal, carbohydrates 54% kcal and fat 14% kcal. The fertility enhancement composition (FE; TD 230256) was calorie-matched to the CON diet and contained 3.0 kcal/g made up by protein 30.2% kcal, carbohydrates 49.4% kcal, and fat 20.5% kcal. The FE also included weight loss agents (green coffee bean extract—2.5 g/kg, green tea extract—0.75 g/kg and forskolin—0.125 g/kg); mitochondrial enhancers (α-lipoic acid—1 g/kg, CoQ10—2.5 g/kg, vitamin E/DL-α tocopheryl acetate—2.07 g/kg and beetroot extract—10 g/kg and creatine monohydrate—30 g/kg); and fertility support agents (L-arginine—5 g/kg, iron/ferric citrate—0.4 g/kg, folic acid—0.005 g/kg, omega-3—28 g/kg, vitamin D3—2.4 mg/kg, vitamin B12—0.05 mg/kg, zinc—80 mg/kg, copper—25 mg/kg, and selenium—0.34 mg/kg). All experiments and procedures were approved by the McMaster University Animals Ethics Committee and conducted under appropriate Canadian guidelines for animal research.

### Results

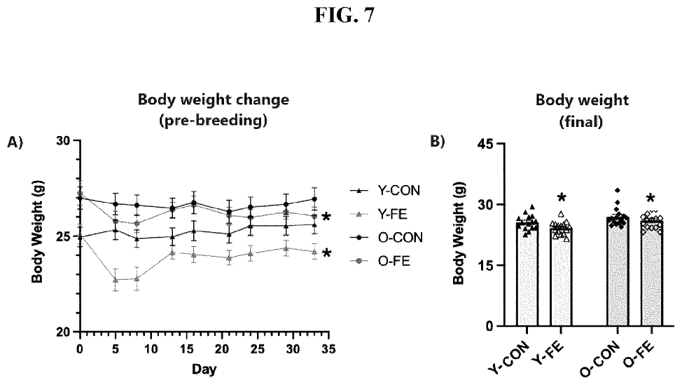

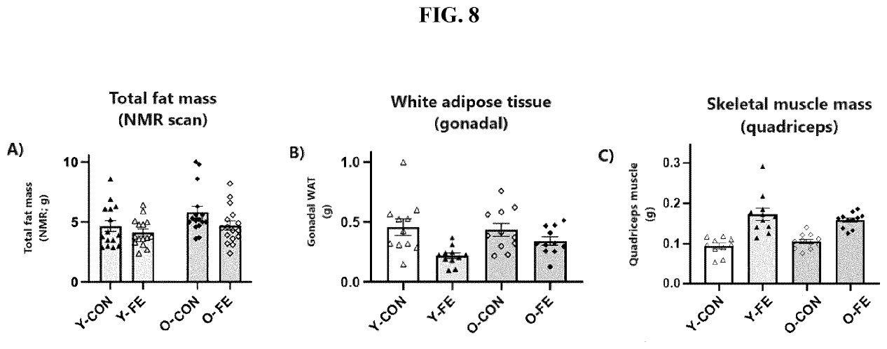

To assess whether the fertility enhancement composition (FE) improved bodyweights, body composition, and organ weights (skeletal muscle and gonadal fat), each were measured at 1 month following dietary intervention and/or at the end of the second breeding cycle. FE improved bodyweight (BW; FIG. 7 A/B), total fat mass (FIG. 8A), gonadal white adipose tissue (WAT; FIG. 8B) and muscle mass (FIG. 8C) in both younger and older mice. These results show that the fertility enhancement composition improves (lowers) bodyweight, (lowers) total fat mass, (lowers) gonadal-specific fat mass, and (increases) muscle mass across age groups.

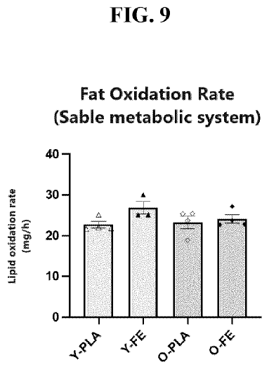

To assess whether the fertility enhancement composition enhanced in vivo fat burning (lipid oxidation), virgin female mice were assessed in metabolic chambers at 1 month following dietary intervention. In vivo fat oxidation was indeed improved in the FE groups in both younger and older mice (FIG. 9), demonstrating that the fertility enhancement composition acts to enhance the rate of fat burning in vivo across age groups.

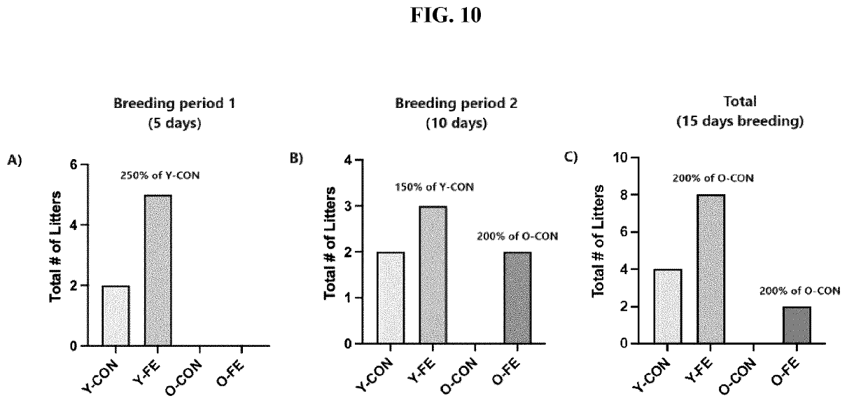

To assess whether the fertility enhancement composition improves female fertility and reproductive success, sperm plugs and litters were counted following two separate harem breeding periods (5 days and 10 days, respectively). Again, surprisingly, number of litters were improved by FE in both young and old female mice (FIG. 10 A-C). As a result, copulatory success (total litters/sperm plugs) was also improved in the FE animals across age groups (Table 4).

TABLE 4
Effect of FE supplementation on mating behavior and reproductive
outcomes in younger and older female mice.

| Mating and Reproductive Outcomes | Y-CON | Y-FE | O-CON | O-FE |
|---|---|---|---|---|
| Copulatory Plugs | 9 | 9 | 7 | 11 |
| Number of Litters | 4 | 8 | 0 | 2 |
| Copulatory Success | 44% | 89% | 0% | 18% |
| ([Litters/Plugs] × 100) | | | | |

These results clearly show that the fertility enhancement composition improved reproductive success and couple fertility in younger and older mice.

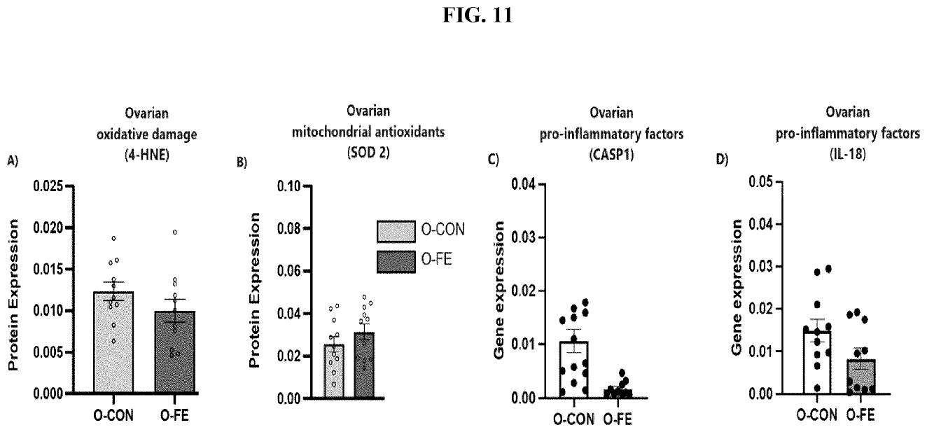

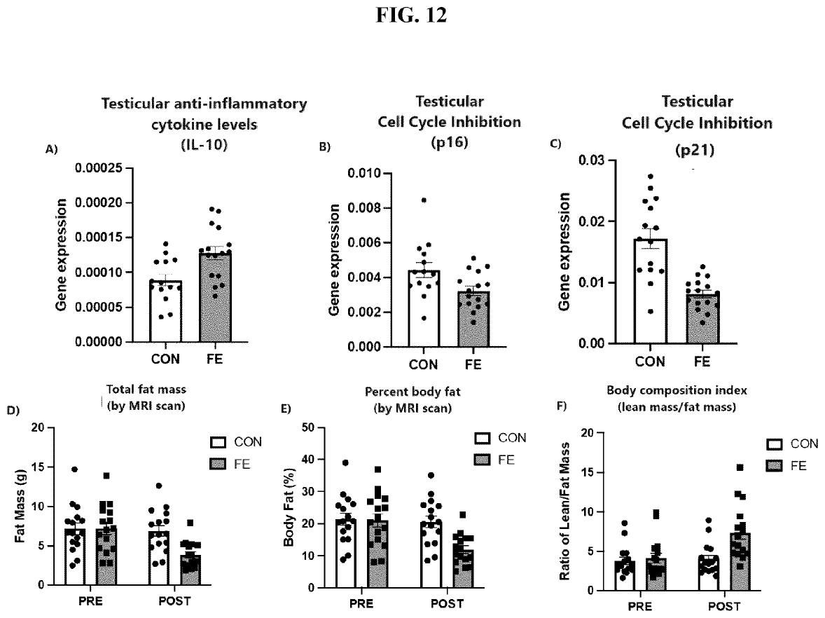

In order to assess whether the fertility enhancement composition improved ovarian pathology, markers of oxidative damage (4-HNE; lipid peroxidation), mitochondrial antioxidant defense (SOD2) and inflammation (CASP and IL-18) were measured in ovaries by immunoblotting and rtPCR. FE significantly improved these pathology markers in older mice (FIG. 11A-D), demonstrating that the fertility enhancement composition mitigates the driving mechanisms of infertility; namely, oxidative damage, mitochondrial dysfunction (reduced antioxidant status), and inflammation.

In summary, these findings clearly show that administration of the fertility enhancement composition: 1) reduces body weight and body fat, 2) improves the number of litters arising from successful mating (copulatory success), and 3) lowers (improves) biochemical markers associated with infertility (oxidative damage, mitochondrial antioxidants and inflammation).

### Example 3—Fertility Enhancement Composition Improves Testicular Pathology Markers in Males

To determine if a fertility enhancement (FE) composition in accordance with an embodiment of the invention, for example as described in Examples 1 and 2, comprising mitochondria-enhancing agents, weight loss agents and fertility-support agents could improve body composition and testicular pathology markers, such as anti-inflammatory factors and cell-cycle arrest, in male mice, the following experiments were conducted.

Following breeding, the young male mice (6 months of age) used for breeding in Experiment 2 were fed either a control (CON, n=16) or Fertility Enhancer (FE, n=16) diet for four weeks, followed by organ harvest. The primary outcomes measured were antioxidant markers (IL-10) and inhibitors of the sperm cell cycle (p16 and p21) in testicular tissue. Secondary outcomes were pre vs. post treatment improvements in body composition, including total fat mass, percent body fat and body composition index (lean mass/fat mass).

### Results

To assess whether the fertility enhancement composition (FE) improved drivers of infertility in males, testicular anti-inflammatory factors (IL-10) and key inhibitors of sperm cell-cycle arrest (p16 and p21) as well as changes in body composition were assessed in younger males following one month of treatment. FE-treated male mice demonstrated A) increased anti-inflammatory IL-10 levels and B-C) reduced cell-cycle arrest markers (p16 and p21) and remarkable improvements in body composition, including improved D) total fat mass, E) percent body fat and F) body composition index (lean mass/fat mass). These data demonstrate that the fertility enhancer composition improves body composition, anti-inflammatory factors and cell-cycle arrest; thus, likely favouring overall health and the maturation of sperm cells/spermatogenesis in testicles of younger males.

In summary, these findings clearly show that administration of the fertility enhancement composition: 1) improves key biochemical indicators of male infertility (low antioxidant status and increased cell-cycle arrest in testicular tissue) and 2) improves all aspects of body composition (fat mass, percent body fat and body composition index).

A composition as described herein for use in primates, including humans, has been determined based in the foregoing, and adjusted accordingly for use in primates using accepted protocols (Nair and Jacob, J Basic and Clin Pharma, 2016; 7:27-31), with additional adjustments to comply with regulatory requirements.

1. A fertility enhancement composition comprising at least one of a weight loss agent, at least one of a mitochondria-enhancing agent and at least one of a fertility support agent.

2. The composition of claim 1, wherein the weight loss agent is selected from the group consisting of psyllium, guar gum, capsaicin, chitosan, caffeine, Garcina cambogia, Pinus densiflora, capsaicin, yohimbe, hoodia, glucomannan, African mango, guarana, pyruvate, carnitine, beta-glucans, fucoxanthin, raspberry ketone, white kidney bean, kola nut, chromium, ginseng, psyllium, St. John's wort, dandelion, hydroxycitric acid, conjugated linoleic acid, green tea extract, black tea extract, green coffee bean extract, forskolin, bitter orange and mixtures thereof.

3. The composition of claim 1, wherein the weight loss agent comprises a mixture of green tea extract, green coffee bean extract and forskolin.

4. The composition of claim 3, wherein the weight loss agent additionally comprises conjugated linoleic acid.

5. The composition of claim 1, wherein the mitochondria-enhancing agent is selected from the group consisting of beetroot extract, nitrates, nicotinamide riboside, vitamin C, vitamin D, vitamin E, thiamine, riboflavin, magnesium, calcium, phosphate, membrane phospholipids, creatine, pyruvate, coenzyme Q10, NADH, nicotinic acid, 1-carnitine, dichloroacetate, curcumin and mixtures thereof.

6. The composition of claim 1, wherein the mitochondria-enhancing agent comprises a mixture of beetroot extract, coenzyme Q10, alpha lipoic acid, creatine monohydrate and vitamin E.

7. The composition of claim 1 where the fertility support comprises a mixture of folate/folic acid, iron, L-arginine, and omega-3 (DHA/EPA).

8. The composition of claim 1, wherein the weight loss agents comprise green tea extract, green coffee bean extract and forskolin and the mitochondria-enhancing agents comprise beet root extract, coenzyme Q10, alpha lipoic acid, creatine monohydrate and vitamin E and the fertility support agents comprise folate/folic acid, iron, L-arginine, selenium, zinc, copper, vitamin D, vitamin B12 and omega-3 fatty acids (DHA/EPA).

9. The composition of claim 8, where the amount of each agent is: 25-1000 mg of green tea extract, 50-1000 mg of green coffee bean extract, 15-100 mg of forskolin, and as the mitochondria-enhancing agent a mixture comprising a daily dosage of 50-5000 mg of beetroot extract, 50-900 mg of coenzyme Q10, 50-900 mg alpha lipoic acid, 1-5 g of creatine monohydrate, and 50-900 IU of vitamin E, and as the fertility support agents; 200-1500 mg each of DHA and EPA as omega-3/fish oil, 1-100 mg of elemental iron as an iron salt, 50-2000 mg of L-arginine, and 200-1000 μg of folate/folic acid.

10. The composition of claims 7-9, additionally comprising the fertility support agents:
iron, L-arginine, vitamin D and vitamin B12, and optionally, zinc, copper and selenium.

11. The composition of claim 10, comprising vitamin D (400-2,000 IU/day, e.g. 500-1500 IU/day, or 1,000 IU/day), vitamin B12 (10-100 μg/day, e.g. 20-80 μg/day, 40-60 μg/day or 50 μg/day), zinc (10-50 mg/day, e.g. 20-40 mg/day or 30 mg/day), copper (1-5 mg/day, e.g. 3 mg/day), and selenium (10-125 ug/day, e.g. 30-100 μg/day, 50-80 μg/day or 70 μg/day).

12. The composition of claim 1, formulated for oral administration.

13. The composition of claim 1, which is provided in at least two different dosage forms.

14. The composition of claim 1, wherein the weight loss agent is provided in a first dosage form and the mitochondria-enhancing agent is provided in a second dosage form.

15. A method for promoting fertility enhancement in a mammal comprising the step of administering to the mammal a fertility enhancement composition as defined in any one of claims 1-14.

16. The method of claim 9, wherein:
the weight loss agent is selected from the group consisting of psyllium, guar gum, capsaicin, chitosan, caffeine, Garcina cambogia, Pinus densiflora, capsaicin, yohimbe, hoodia, glucomannan, African mango, guarana, pyruvate, carnitine, beta-glucans, fucoxanthin, raspberry ketone, white kidney bean, kola nut, chromium, ginseng, psyllium, St. John's wort, dandelion, hydroxycitric acid, conjugated linoleic acid, green tea extract, black tea extract, green coffee bean extract, forskolin, bitter orange and mixtures thereof:
the mitochondria-enhancing agent is selected from the group consisting of beetroot extract, nitrates, nicotinamide riboside, vitamin C, vitamin D, vitamin E, thiamine, riboflavin, magnesium, calcium, phosphate, membrane phospholipids, creatine, pyruvate, coenzyme Q10, NADH, nicotinic acid, l-carnitine, curcumin and mixtures thereof; and
the fertility support agent is selected from: folate, folic acid, folinic acid, ferrous salts, such as ferrous gluconate, ferrous sulfate, ferrous fumarate or ferrous bisglycinate, omega 3 fatty acids (either as fish oil or individual EPA and DHA supplements), L-arginine, L-citrulline and mixtures thereof.

17. The method of claim 9, wherein the weight loss agent comprises a mixture of green tea extract, green coffee bean extract and forskolin; the mitochondria-enhancing agent comprises a mixture of beetroot extract, coenzyme Q10, alpha lipoic acid, creatine monohydrate and vitamin E; and the fertility support agent comprises folate/folic acid, iron, L-arginine, and omega-3 (DHA/EPA).

18. The method of claim 9, wherein the composition additionally comprises the fertility support agents: iron, L-arginine, vitamin D and vitamin B12, and optionally, zinc, copper and selenium.

19. The method of claim 9, wherein the composition is administered once daily.

20. The method of claim 9, wherein the composition is administered in portions two or more times daily.

21. The method of claim 9, wherein the weight loss agent is administered in a first dosage form and the mitochondria-enhancing agent is administered in a second dosage form.

22. The method of claim 9, wherein the mammal is obese or overweight.

23. The method of claim 9, wherein fertility enhancement includes reduction of body weight and/or body fat with no reduction or an increase in lean body mass.

24. A method to treat at least one of the following in a mammal: infertility, body weight, body fat, body composition index, mitochondrial capacity, fatty liver disease, dyslipidemia, oxidative stress levels, gonadal apoptosis/atrophy, and systemic and/or gonadal inflammation levels comprising administering to the mammal a composition as defined in any one of claims 1-14.

25. A method of treating infertility in a mammalian male and female couple comprising:
i) administering to the male of the couple a fertility-enhancement composition as defined in any one of claims 1-14;
ii) administering to the female of the couple a fertility-enhancement composition as defined in any one of claims 1-14; or
iii) administering to both the male and female of the couple a fertility-enhancement composition as defined in any one of claims 1-14.
